(19) United States
(12) Patent Application Publication
(10) Pub. No.: US 2022/0117890 A1
(43) Pub. Date: Apr. 21, 2022

(54) CANNABINOID EMULSIONS

(71) Applicant: CORN PRODUCTS DEVELOPMENT, INC., Westchester, IL (US)

(72) Inventors: DEREK HOLTHAUS, BRIDGEWATER, NJ (US); ROBERT LUPITSKYY, BRIDGEWATER, NJ (US); SCOTT MAGNESS, BRIDGEWATER, NJ (US)

(21) Appl. No.: 17/505,984

(22) Filed: Oct. 20, 2021

Related U.S. Application Data
(60) Provisional application No. 63/094,520, filed on Oct. 21, 2020.

(51) Int. Cl. A61K 9/107; A61K 47/36; A61K 47/44; A61K 9/00; A61K 31/352; A61K 31/047; A23D 7/005; A23D 7/04; A23L 33/105; A23L 33/115; A23L 2/52

(52) U.S. Cl. CPC A61K 9/1075; A61K 47/36; A61K 47/44; A61K 9/0095; A61K 31/352; A23V 2002/00; A23D 7/0053; A23D 7/04; A23L 33/105; A23L 33/115; A23L 2/52; A61K 31/047

(57) ABSTRACT

The technology disclosed in this specification pertains to an emulsion comprising (i) a continuous aqueous phase, (ii) an disperse oil phase comprising a cannabinoid, and (iii) an emulsifier which is gum arabic, wherein the weight ratio of gum arabic to said disperse oil phase is ≥1:1. The emulsion may have a relatively small particle size with a relatively high oil load in combination. The emulsion may also have a small particle size without requiring a polysorbate or any co-surfactant.

The technology disclosed in this specification pertains to to emulsions comprising a cannabinoid and gum arabic, methods for producing said emulsions and uses of said emulsions.

Cannabinoids are compounds found in cannabis. The best-known cannabinoids are tetrahydrocannabinol (THC) and cannabidiol (CBD). Other cannabinoids include cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), THCP (tetrahydrocannabiphorol), cannabidivarin (CBDV), cannabichromevarin (CBCV), cannabigerovarin (CBGV), cannabigerol monomethyl ether (CBGM), tetrahydrocannabinolic acid (THCA), cannabidiolic acid (CBDA) and related compounds. Furthermore, cannabinoid compounds may include synthetic cannabinoids. These are generally molecules which are based on the structure of herbal cannabinoids.

Cannabidiol (CBD) oil extracted from hemp and marijuana (Cannabis sativa) is of interest due to its perceived health benefits which range from pain relief to anxiety suppression and beyond. Therefore, there is increased interest in incorporating CBD into foodstuffs to provide the aforementioned health benefits to consumers in an easily deliverable form (foodstuff). However, there are several key challenges associated with adding CBD oils into foodstuffs. First, CBD oils are not water soluble and thus cannot be homogenously incorporated into foodstuffs which are primarily water-based (e.g. beverages). In the case of a beverage, if CBD oil were added into an existing formulation, the oil would float to the top and not evenly distributed throughout the beverage. This creates a significant challenge for dosing and quantification of CBD, as the top portion of the beverage would contain the entirety of the CBD while the rest of the beverage would not contain any. Also, CBD oil generally exhibits low oral bioavailability, as the digestive enzymes and other biological processes can only partially (and slowly) digest CBD oil and transport the CBD to the bloodstream. CBD also is very slow to reach the bloodstream upon oral administration, and thus there is a significant need to speed up the delivery.

The class cannabinoid further comprises other compounds which exhibit various effects. THC is for example the cannabinoid which is the primary psychoactive compound in cannabis.

Gum arabic is a known emulsifier used in a wide variety of foods.

Gum arabic may be from Acacia senegal or Acacia seyal. Gum arabic from Acacia senegal is most commonly used for emulsions.

When using emulsions comprising cannabinoid in the disperse phase, it is believed that a smaller droplet size, and thus a larger surface area, may increase the digestive enzyme function and therefore increases oral bioavailability and time to onset, as well as reduces the required dosing of the cannabinoid to achieve a desired result.

In view of the above, there is a need for an improved emulsion comprising a cannabinoid enabling to combine a relatively small particle size of the disperse oil phase with a relatively high oil load.

Furthermore, there is a need for an improved emulsion comprising a cannabinoid wherein a relatively small particle size of the disperse oil phase may be obtained without requiring polysorbate or any co-surfactant.

In an aspect, the technology disclosed in this specification pertains to an emulsion comprising (i) a continuous aqueous phase, (ii) an disperse oil phase comprising a cannabinoid, and (iii) an emulsifier which is gum arabic, wherein the weight ratio of gum arabic to said disperse oil phase is ≥1:1.

In an aspect, the technology disclosed in this specification pertains further provides a beverage comprising the emulsion as described in this specification.

In an aspect, the technology disclosed in this specification pertains further provides a method of preparing a beverage, said method comprising incorporating and/or admixing the emulsion as described in this specification into said beverage. The technology disclosed in this specification pertains to a beverage obtainable by this method.

In an aspect, the technology disclosed in this specification pertains further provides an emulsion or beverage as described in this specification, for use as a medicament.

In an aspect, the technology disclosed in this specification pertains provides an emulsion comprising (i) a continuous aqueous phase, (ii) an disperse oil phase comprising a cannabinoid, and (iii) an emulsifier which is gum arabic, wherein the weight ratio of gum arabic to said disperse oil phase is ≥1:1.

As will be understood by the skilled person, the emulsion is an oil-in-water emulsion, wherein oil phase droplets are dispersed within the aqueous continuous phase.

In any embodiment described in this specification, the weight ratio of gum arabic to said disperse oil phase is ≥1:1. The skilled person will understand that as used herein the weight of the oil phase refers to the sum weight of the components present in the disperse oil phase, excluding emulsifiers present in the emulsion.

In any embodiment described in this specification, the weight ratio of gum arabic to said disperse oil phase is ≥1.5:1, or ≥2:1, or ≥3:1, or ≥4:1. It has been found that an increased weight ratio of gum arabic to the disperse oil phase has the advantage of enabling a larger weight percentage of the disperse oil phase in the emulsion. Furthermore, it has been found that an increased weight ratio of gum arabic to the disperse oil phase has the advantage of enabling a smaller median particle size (d50) without requiring a co-surfactant such as polysorbate.

There is no specific upper limit for the weight ratio of gum arabic to the disperse oil phase. The weight ratio of gum arabic to the disperse oil phase may for instance be ≤6:1, or ≤5:1.

The weight fraction of the disperse oil phase in the emulsion may vary within wide limits. The weight fraction of the disperse oil phase in the emulsion may for instance be from 1 to 20 wt. % based on the weight of the emulsion, or from 2 to 15 wt. % based on the weight of the emulsion.

The disperse oil phase may have any suitable particle size. In any embodiment described in this specifciation, the disperse oil phase has a median particle size (d50) of 500 nm or less, or from 100 to 400 nm, more or from 150 to 300 nm. As used herein the median particle size (d50) is determined by a Malvern apparatus as described in the section “measurement methods”

In any embodiment described in this specification, the weight fraction of the disperse oil phase in the emulsion is from 2 to 8 wt. % based on the weight of the emulsion, more or from 3 to 6 wt. % based on the weight of the emulsion. Applying a weight fraction within these ranges was found to facilitate reducing the particle size of the disperse phase. In any embodiment described in this specifciation, a weight fraction of the disperse oil phase within these ranges is combined with a weight ratio of gum arabic to the disperse oil phase of ≥3:1 or even more or ≥4:1. This is found to enable an even further reduction in particle size of the disperse phase, such as a median particle size (d50) of 250 nm or less, or from 100 to 200 nm, or from 150 to 200 nm.

In any embodiment described in this specification, the weight fraction of the disperse oil phase in the emulsion from 8 to 20 wt. % based on the weight of the emulsion, or from 10 to 15 wt. % based on the weight of the emulsion. In any embodiment described in this specification, a weight fraction within these ranges is combined with a weight ratio of gum arabic to the disperse oil phase of ≥1:1 and ≤3:1, or ≤2.5:1. The disperse oil phase in this embodiment may advantageously have a median particle size (d50) from 200 to 500 nm, or from 250 to 400 nm.

The disperse oil phase may comprise any suitable cannabinoid. In any embodiment, the cannabinoid is selected from the group consisting of tetrahydrocannabinol (THC) and cannabidiol (CBD). In any embodiment the cannabinoid may also be THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), or CBT (cannabicitran).

The disperse oil phase may comprise a vegetable oil. The cannabinoid may be admixed with and/or dissolved in the vegetable oil. The vegetable oil may be any triglyceride oil extracted from seeds. Any suitable vegetable oil may be used, for instance a vegetable oil selected from the group consisting of medium chain triglyceride (MCT) oil, coconut oil, corn oil, cottonseed oil, olive oil, palm oil, peanut oil, rapeseed oil, safflower oil, sesame oil, soybean oil, sunflower oil, and canola oil. Generally, a vegetable oil has a density below that of water, hence below 1.0 g/ml.

It was found that the presence of a vegetable oil as disclosed hereinabove facilitates obtaining a stable emulsion in the event the cannabinoid has a low density. Without wishing to be bound by any scientific theory, it is believed that a vegetable oil having a density between the density of a cannabinoid having a low density and the density of water may assist to minimize the density difference between the disperse and continuous phase, thereby further enhancing the stability of the emulsion.

Based on the teaching provided herein, the skilled person is able to determine suitable ratios between the cannabinoid and the vegetable oil. The weight ratio of the cannabinoid and the vegetable oil may be between 1:0.1 and 1:9, for instance between 1:3 and 3:1. As used herein the weight of the cannabinoid refers to the sum weight of all cannabinoids which may be present in the disperse oil phase.

Any suitable gum arabic may be used. The gum arabic may be gum arabic from Acacia senegal or from Acacia seyal. In any embodiment described in this specification, the gum arabic is gum arabic from Acacia senegal.

It is possible to use gum arabic having an increased molecular weight, such as for instance gum arabic as disclosed in EP-A-1 611 159, EP-A-1 666 502 or gum arabic as disclosed in PCT application no PCT/US20/45447 filed on Aug. 7, 2020, the contents of which documents are incorporated in the present application by reference in its entirety. However, this is not necessary.

The emulsion may comprise further emulsifiers in addition to gum arabic. However, this is not necessary. In any embodiment described in this specification, gum arabic is the sole emulsifier in the emulsion. It was found that the presence of non-natural emulsifier such as polysorbate may negatively impact the bioavailability of the cannabinoid. In any embodiment described in this specification the emulsion does not comprise a polysorbate.

The emulsion may optionally contain any suitable optional additive, for instance a preservative. Exemplary additives which may be present in the emulsion include an acid, or an organic acid, for instance citric acid and/or ascorbic acid, potassium sorbate and/or sodium benzoate.

The emulsion can be prepared using methods known in the art. In any embodiment may be prepared by a process comprising:

i. providing an aqueous phase comprising water and an emulsifier which is gum arabic;
ii. providing an oil phase comprising an oil and a cannabinoid extract;
iii. mixing said aqueous phase and said oil phase to create a pre-emulsion; and
iv. homogenizing said pre-emulsion to obtain the emulsion.

In any embodiment described in this specification, the said homogenizing comprises high pressure homogenization at a pressure of at least 130 bar, or between 240 and 2100 bar. In any embodiment described in this specification, the homogenizing is affected in using a microfluidizer. In any embodiment described in this specificaiton, homogenizing is affected using at least 2 passes.

In an aspect, the technology disclosed in this specification pertains to a beverage comprising the emulsion as described in this specification.

In an aspect, the technology disclosed in this specification pertains to a method of preparing a beverage, said method comprising incorporating and/or admixing the emulsion as described in this specification into said beverage. In an aspect, the technology disclosed in this specification pertains to a beverage obtainable by this method.

In an aspect, the technology disclosed in this specification pertains to an emulsion or beverage as described in this specification, for use as a medicament.

The technology disclosed in this specification can be better understood with reference to the following aspects, which are not intended to limit the full scope of the invention.

An emulsion comprising (i) a continuous aqueous phase, (ii) an disperse oil phase comprising a cannabinoid, and (iii) an emulsifier which is gum arabic, wherein the weight ratio of gum arabic to said disperse oil phase is ≥1:1

The emulsion according to claim 1, wherein the weight ratio of gum arabic to said disperse oil phase is ≥1.5:1, or ≥2:1

The emulsion according to claim 2, wherein the weight ratio of gum arabic to said disperse oil phase is ≥3:1, or ≥4:1.

The emulsion according to any preceding claim, wherein the weight ratio of gum arabic to said disperse oil phase is ≤6:1, or ≤5:1.

The emulsion according to any preceding claim, wherein the weight fraction of the disperse oil phase in the emulsion is from 1 to 20 wt. % based on the weight of the emulsion, or from 2 to 15 wt. % based on the weight of the emulsion.

The emulsion according to any preceding claim, wherein said disperse oil phase has a median particle size (d50) of 500 nm or less, or from 100 to 400 nm, or from 150 to 300 nm.

The emulsion according to any preceding claim, wherein the weight fraction of the disperse oil phase in the emulsion is from 2 to 8 wt. % based on the weight of the emulsion, or from 3 to 6 wt. % based on the weight of the emulsion.

The emulsion according to claim 7, wherein the weight ratio of gum arabic to said disperse oil phase is ≥3:1, or ≥4:1.

The emulsion according to claim 7 or 8, wherein said disperse oil phase has a median particle size (d50) of 250 nm or less, or from 100 to 200 nm, more or from 150 to 200 nm.

The emulsion according to any one of claims 1 to 6, wherein the weight fraction of the disperse oil phase in the emulsion is from 8 to 20 wt. % based on the weight of the emulsion, or from 10 to 15 wt. % based on the weight of the emulsion.

The emulsion according to claim 10, wherein the weight ratio of gum arabic to said disperse oil phase is ≤3:1, or ≤2.5:1.

The emulsion according to claim 10 or 11, wherein said disperse oil phase has a median particle size (d50) from 200 to 500 nm, or from 250 to 400 nm.

The emulsion according to any preceding claim, wherein said cannabinoid is a) one or more of tetrahydrocannabinol (THC) and cannabidiol (CBD) or is b) selected from the group consisting of tetrahydrocannabinol (THC) and cannabidiol (CBD) cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), THCP (tetrahydrocannabiphorol), cannabidivarin (CBDV), cannabichromevarin (CBCV), cannabigerovarin (CBGV), cannabigerol monomethyl ether (CBGM), tetrahydrocannabinolic acid (THCA), cannabidiolic acid (CBDA) or mixtures thereof.

The emulsion according to any preceding claim, wherein, the disperse oil phase comprises a vegetable oil.

The emulsion according to preceding claim, wherein said vegetable oil is selected from the group consisting of medium chain triglyceride (MCT) oil, coconut oil, corn oil, cottonseed oil, olive oil, palm oil, peanut oil, rapeseed oil, safflower oil, sesame oil, soybean oil, sunflower oil and canola oil.

The emulsion according to any preceding claim, wherein the weight ratio of said cannabinoid to vegetable oil is between 1:0.1 and 1:9, for instance between 1:3 and 3:1.

The emulsion according to any preceding claim, wherein said gum arabic is the sole emulsifier in the emulsion.

A beverage comprising the emulsion according to any preceding claim.

A method of preparing a beverage, said method comprising incorporating and/or admixing the emulsion according to any preceding claim into said beverage.

Beverage obtainable by the method according to claim 16.

Emulsion or beverage according to any preceding claim, for use as a medicament.

Method for preparing an emulsion according to any one of claims 1 to 15, said method comprising: providing an aqueous phase comprising water and an emulsifier which is gum arabic, providing an oil phase comprising an oil and a cannabinoid extract, mixing said aqueous phase and said oil phase to create a pre-emulsion; and homogenizing said pre-emulsion to obtain the emulsion.

The technology disclosed in this specification can be better understood with reference to the following examples, which are not intended to limit the full scope of the invention.

### Measurement Methods

Turbiscan Stability Index (TSI)

TSI is a parameter developed specially for formulators to rapidly compare and characterize the physical stability of various formulations and is measured using a Turbiscan Lab Expert (Formulaction) and software TurbiSoft-2.0.0.19. Regarding any embodiment described in this specification, TSI is used to monitor the physical stability of the nanoemulsion concentrate. Any destabilization phenomenon that occurs in a sample will have an impact on the backscattering signal intensities over time. The formulation with the largest change in backscattering intensity is the least stable and has the highest TSI. The calculation of TSI is as follows:

$$TSI = \frac{{Σ}_{h} \vert {scan}_{i} ( h ) - {scan}_{i - 1} ( h ) \vert}{H}$$

where the TSI calculation sums up the evolution of backscattered light at all measured position (h), based on a scan-to-scan difference, over total sample height (H).

Turbiscan vials (Formulaction) are filled 4 cm high with each emulsion concentrate and are measured for backscattering several times over a period of 21 days. At day 21, the TSI (Global) is recorded and the emulsion concentrates can be compared against each other for stability against destabilization phenomenon. Larger TSI values correspond to less stable emulsion concentrates.

### Emulsifying Ability Median

The median particle size (d50), as well as d10, d90, d[4,3] were measured using a particle size analyzer (Manufacturer: Malvern; Model: Mastersizer 2000).

### EXAMPLES

### Examples 1-6, Reference Experiment A

All examples and comparative experiments described herein involved the use of gum arabic from Acacia senegal (TIC Pretested® Gum Arabic Spray Dry Powder).

Nanoemulsions comprising gum arabic as the emulsifier and CBD isolate powder ((>98% purity) purchased from Treehouse Biotech (Longmont, Colo.) as the cannabinoid source were prepared according to the following formulations (Table 1). All percentages are given in wt. %.

TABLE 1

| | Gum | MCT | CBD | Citric | Ascorbic | Potassium | Sodium | | Ratio Gum |
|---|---|---|---|---|---|---|---|---|---|
| | arabic | Oil | Isolate | acid | acid | Sorbate | Benzoate | Water | arabic:oil |
| Example | (%) | (%) | (%) | (%) | (%) | (%) | (%) | (%) | phase |
| 1 | 20.0 | 3.0 | 2.0 | 0.2 | 0.2 | 0.1 | 0.1 | 74.4 | 5:1 |
| 2 | 25.0 | 3.0 | 2.0 | 0.2 | 0.2 | 0.1 | 0.1 | 69.4 | 5:1 |
| 3 | 20.0 | 6.0 | 4.0 | 0.2 | 0.2 | 0.1 | 0.1 | 69.4 | 2:1 |
| 4 | 25.0 | 6.0 | 4.0 | 0.2 | 0.2 | 0.1 | 0.1 | 64.4 | 2.5:1 |
| 5 | 20.0 | 7.2 | 4.8 | 0.2 | 0.2 | 0.1 | 0.1 | 67.4 | 1.67:1 |
| 6 | 20.0 | 7.2 | 4.8 | 0.4 | — | — | 0.05 | 67.55 | 1.67:1 |
| A (ref) | 10.0 | 12.0 | 8.0 | 0.2 | 0.2 | 0.1 | 0.1 | 69.4 | 0.5:1 |

Citric acid, ascorbic acid, sodium benzoate and potassium sorbate were dissolved in room temperature deionized water via overhead mixing for 5 minutes. The gum arabic was added to the solution and allowed to mix for 30 minutes. Simultaneously in a separate beaker, the MCT (medium chain triglyceride) oil was heated on a hot plate to 65° C. The CBD isolate powder was added to the MCT oil and mixed (via magnetic stirrer bar) until fully dissolved. The CBD oil solution was allowed to cool to room temperature.

A pre-emulsion was made by adding the oil phase into the aqueous phase under high shear mixing conditions (10,000 rpm) for 2 minutes in a homogenizer (Manufacturer: Ross, Model: HSM-LCI-T).

The pre-emulsion was further processed using a Microfluidizer (Microfluidics, Model: M-110EH). The interaction chamber used was the F12Y-H30Z. The mixtures were processed at a pressure of 10000 PSI, or 689.48 bar.

The particle size of the emulsion is immediately tested using a laser diffraction particle size analyzer (Manufacturer: Malvern Mastersizer 2000) where the median particle size (d50), as well as d10, d90, and d[4,3] are recorded. For emulsions with a median particle size (d50) under 100 nm, particle size measurements were taken by dynamic light scattering (DLS) using a Malvern Zetasizer Nano-S and reported as Z-average particle size. Table 2 shows the particle sizes taken after various passes.

The emulsions obtained in examples 1 and 2 have were found to have a median particle size (d50) of below 200 nm. The emulsions obtained in examples 3 to 6 were found to have a median particle size (d50) below 400 nm for emulsions wherein the weight fraction of the disperse phase was as high as 12 wt. %.

TABLE 2

| Example | Pass | d10 (nm) | d50 (nm) | d90 (nm) | d[4, 3] (nm) |
|---|---|---|---|---|---|
| 1 | 1 | 151.85 | 235.91 | 349.18 | 246.11 |
| | 3 | 106.86 | 172.19 | 255.62 | 177.95 |
| | 5 | 108.21 | 175.55 | 262.26 | 181.63 |
| 2 | 1 | 119.54 | 198.88 | 302.51 | 206.44 |
| | 3 | 100.5 | 158.88 | 236.09 | 164.32 |
| | 5 | 98.78 | 156.51 | 232.65 | 161.99 |
| 3 | 1 | 144.96 | 247.24 | 375.06 | 256.15 |
| | 3 | 186.15 | 296.68 | 422.28 | 301.51 |
| | 5 | 227.7 | 349.33 | 486.59 | 354.14 |
| 4 | 1 | 152.65 | 255.57 | 377.67 | 262.04 |
| | 2 | 190.59 | 281.43 | 387.54 | 287.07 |
| | 3 | 135.3 | 226.18 | 335.35 | 232.02 |
| 5 | 1 | 188.33 | 305.92 | 462.69 | 319.55 |
| | 2 | 201.77 | 326.5 | 478.85 | 335.09 |
| | 3 | 238.02 | 378.62 | 539.93 | 385.66 |
| 6 | 2 | 229.5 | 372.11 | 557.76 | 385.48 |
| A (Ref) | 1 | 558.5 | 983.43 | 1352.70 | 975.42 |
| | 2 | 676.56 | 993.13 | 1307.28 | 991.22 |
| | 3 | 687.69 | 994.09 | 1305.77 | 994.66 |

### Examples 1.a-6.a, Reference Experiment A.a

Beverage stability was evaluated by diluting the nanoemulsions, obtained after the final pass, to a CBD content of 25 mg per 355 g of beverage, such as according to Table 3. The citric acid and sodium benzoate are added to room temperature deionized water and mixed via magnetic stir bar for 5 minutes. The CBD nanoemulsion is added to the solution and lightly mixed. A 12 oz bottle is filled with the solution and capped. The bottle is store horizontally at room temperature without manipulation for 21 days. After 21 days, the beverage is visually examined without manipulation for the presence of a white ring at the top of the beverage (creaming of the CBD emulsion). The beverage can also be examined for sedimentation.

TABLE 3

| Ingredient | % (w/w) |
|---|---|
| Emulsion (2% CBD isolate; 4%; 4.8%; 8%) | 0.35; 0.7; 0.84; 1.4 |
| Sodium Benzoate | 0.1 |
| Citric acid | 0.3 |
| Water | Balance |

Table 4 provides the beverage stability and TSI of the emulsions.

TABLE 4

| Example | Beverage Stability | TSI (Global) |
|---|---|---|
| 1.a | Stable | 0.9 |
| 2.a | Stable | 1.3 |
| 3.a | Stable | 0.8 |
| 4.a | Stable | 0.5 |
| 5.a | Stable | 0.9 |
| 6.a | Stable | 0.8 |
| A.a | Not Stable | 2.7 |

### Reference Experiment B

A nanoemulsion using polysorbate 80 as the emulsifier was prepared according to Table 5. The polysorbate nanoemulsion was processed at a pressure of 30000 PSI. The particle size measurement was taken by dynamic light scattering (DLS) using a Malvern Zetasizer Nano-S and reported as Z-average particle size. The results have been presented in Table 6.

TABLE 5
Experiment B

| Polysorbate | MCT | CBD | Citric | Ascorbic | Potassium | Sodium | |
|---|---|---|---|---|---|---|---|
| 80 | Oil | Isolate | Acid | Acid | Sorbate | Benzoate | Water |
| (%) | (%) | (%) | (%) | (%) | (%) | (%) | (%) |
| 25.0 | 7.5 | 5.0 | 0.2 | 0.2 | 0.1 | 0.1 | 61.9 |

TABLE 6
Experiment B

| | d10 | d50 | d90 | Mean | Beverage | TSI |
|---|---|---|---|---|---|---|
| Pass | (nm) | (nm) | (nm) | (nm) | Stability | (Global) |
| 1 | 84.44 | 133.34 | 199.07 | 138.59 | — | — |
| 3 | — | — | — | 42.24 (Z-avg) | — | — |
| 4 | — | — | — | 39.78 (Z-avg) | Stable | 24.5 |

### Bioavailability

The bioavailability of the emulsions B, 2 and 5 was determined, and compared to single intravenous cannabidiol (CBD solid) and non-emulsified oil (CBD in MCT oil).

Plasma pharmacokinetics following single intravenous cannabidiol (CBD solid) or oral administration (4 emulsions+1 non-emulsified oil) was investigated in male Sprague-Dawley rats. Rats were used for this study because they are an accepted model for characterization of pharmacokinetics of formulations being developed for humans. Twelve (12) single-catheterized rats (275-300 g, jugular vein catheter) were obtained from Envigo and divided into 6 groups of 2 animals each. Rats were acclimated for 5 days; the temperature was controlled from 68-79° F., the humidity was controlled from 20% to 70% and the light source was fluorescent lamps with a light/dark cycle of 12/12 hours on/off. No concurrent medication was administered during the study, and all rats had access (ad-libitum) to Tekland Rodent Chow 2018 (Envigo) and tap water throughout the live phase. All animals were randomly placed. Post acclimation, all animals received a single IV (tail vein injection) or oral treatment of one of the nanoemulsions, a non-emulsified oil, or a positive control based on Table 7. Group 1 animals received a single IV injection while Groups 2-6 received a single oral administration. The non-emulsified oil is CBD isolate dissolved in MCT oil. WFI=sterile water for injection. Dosing and dose volume are in mg/mg (body weight) and mL/kg (body weight) respectively.

TABLE 7

| Emulsion/ | | CBD Dose | Dose Volume | |
|---|---|---|---|---|
| product | Vehicle | (mg/kg) | (mL/kg) | Route |
| CBD solid | 20% DMSO + | 2 | 5 | IV |
| | WFI | | | |
| Non-emulsified | MCT Oil | 10 | 10 | PO |
| oil | | | | |
| B | WFI | 10 | 10 | PO |
| 2 | WFI | 10 | 10 | PO |
| 5 | WFI | 10 | 10 | PO |

The CBD concentration in blood plasma samples obtained at various collection times (15, 30, 60, 120, 180, 240, 480 minutes and 24 hr) was determined. The results are reported in Table 8 below.

TABLE 8

| Sample | Time (min) | Concentration (ng/mL) |
|---|---|---|
| CBD Isolate | Pre-Dose | ND |
| | 5 | 1560.0 |
| | 30 | 481.0 |
| | 60 | 341.0 |
| | 120 | 178.0 |
| | 180 | 89.2 |
| | 240 | 44.2 |
| | 480 | 10.6 |
| | 24 hr | 5.2 |
| Non-emulsified oil | Pre-Dose | ND |
| | 5 | ND |
| | 30 | ND |
| | 60 | ND |
| | 120 | BLQ |
| | 180 | BLQ |
| | 240 | 6.6 |
| | 480 | 13.6 |
| | 24 hr | BLQ |
| B | Pre-Dose | ND |
| | 5 | 86.2 |
| | 30 | 185.0 |
| | 60 | 310.0 |
| | 120 | 131.0 |
| | 180 | 67.7 |
| | 240 | 45.1 |
| | 480 | 21.6 |
| | 24 hr | 5.4 |
| 2 | Pre-Dose | ND |
| | 5 | 107.0 |
| | 30 | 583.0 |
| | 60 | 262.0 |
| | 120 | 103.0 |
| | 180 | 85.8 |
| | 240 | 66.9 |
| | 480 | 35.5 |
| | 24 hr | 5.0 |
| 5 | Pre-Dose | ND |
| | 5 | 87.5 |
| | 30 | 294.0 |
| | 60 | 270.0 |
| | 120 | 142.0 |
| | 180 | 88.2 |
| | 240 | 71.7 |
| | 480 | 48.5 |
| | 24 hr | BLQ |

The following pharmacokinetic parameters were determined: Bioavailability absolute (Fabs), Bioavailability relative to non-emulsified (Frel), the time at maximum concentration, the maximum concentration, and half-life (T_{1/2}). See Table 9.

TABLE 9

| | | | | | | AUClast | AUC_{inf} |
|---|---|---|---|---|---|---|---|
| Sample | F_{abs} | Fᵣₑₗ | Tₘₐₓ | Cₘₐₓ | T_{1/2} | (ng*h/mL) | (ng*h/mL) |
| CBD solid | | | 0.25 | 1560 | N.D. | 1984 | 2013 |
| Non-emulsified oil | 2.04% | 100% | 8.0 | 13.6 | N.D. | 202 | 276 |
| B | 9.01% | 443% | 1.0 | 310 | 6.89 | 894 | 948 |
| 2 | 12.0% | 589% | 0.5 | 583 | 5.43 | 1190 | 1230 |
| 3 | 8.48% | 416% | 0.5 | 294 | 6.11 | 841 | 1270 |

It can thus be observed that the nanoemulsions achieved maximum concentration faster compared to the polysorbate and non-emulsified oil. They further achieved much higher bioavailability than the emulsified oil.

1. An emulsion comprising (i) a continuous aqueous phase, (ii) an disperse oil phase comprising a cannabinoid, and (iii) an emulsifier which is gum arabic, wherein the weight ratio of gum arabic to said disperse oil phase is at least 1:1.

2. The emulsion according to claim 1, wherein the weight ratio of gum arabic to said disperse oil phase is at least 1.5:1.

3. The emulsion according to claim 1, wherein the weight ratio of gum arabic to said disperse oil phase is at least 3:1.

4. The emulsion according to claim 1, wherein the weight ratio of gum arabic to said disperse oil phase is from 1.1 to 6:1.

5. The emulsion according to claim 1, wherein the weight fraction of the disperse oil phase in the emulsion is from 1 to 20 wt. % based on the weight of the emulsion.

6. The emulsion according to claim 1, wherein said disperse oil phase has a median particle size (d50) of 500 nm.

7. The emulsion according to claim 1, wherein the weight fraction of the disperse oil phase in the emulsion is from 2 to 8 wt. % based on the weight of the emulsion; wherein the weight ratio of gum arabic to said disperse oil phase is greater than 3:1; and wherein the disperse oil phase has a median particle size (d50) of 250 nm or less.

8. (canceled)

9. (canceled)

10. The emulsion according to claim 1, wherein the weight fraction of the disperse oil phase in the emulsion is from 8 to 20 wt. % based on the weight of the emulsion; wherein the weight ratio of gum arabic to said disperse oil phase is from 1:1 to 3:1; wherein said disperse oil phase has a median particle size (d50) from 200 to 500 nm.

11. (canceled)

12. (canceled)

13. The emulsion according to claim 1, wherein said cannabinoid is
a) one or more of tetrahydrocannabinol (THC) and cannabidiol (CBD) or is
b) selected from the group consisting of tetrahydrocannabinol (THC) and cannabidiol (CBD) cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), THCP (tetrahydrocannabiphorol), cannabidivarin (CBDV), cannabichromevarin (CBCV), cannabigerovarin (CBGV), cannabigerol monomethyl ether (CBGM), tetrahydrocannabinolic acid (THCA), cannabidiolic acid (CBDA) or mixtures thereof.

14. The emulsion according to claim 1, wherein, the disperse oil phase comprises a vegetable oil.

15. The emulsion according claim 1, wherein said vegetable oil is selected from the group consisting of medium chain triglyceride (MCT) oil, coconut oil, corn oil, cottonseed oil, olive oil, palm oil, peanut oil, rapeseed oil, safflower oil, sesame oil, soybean oil, sunflower oil and canola oil.

16. The emulsion according to claim 1, wherein the weight ratio of said cannabinoid to vegetable oil is between 1:0.1 and 1:9.

17. The emulsion according to claim 1, wherein said gum arabic is the sole emulsifier in the emulsion.

18. (canceled)

19. A method comprising admixing an emulsion comprising (i) a continuous aqueous phase, (ii) an disperse oil phase comprising a cannabinoid, and (iii) an emulsifier which is gum arabic, wherein the weight ratio of gum arabic to said disperse oil phase is greater than 1:1 and a second ingredient to make a beverage.

20. (canceled)

21. (canceled)

22. A method comprising:
i. providing an aqueous phase comprising water and an emulsifier which is gum arabic
ii. providing an oil phase comprising an oil and a cannabinoid extract,
iii. mixing said aqueous phase and said oil phase to create a pre-emulsion; and
iv. homogenizing said pre-emulsion to obtain the emulsion.
wherein the weight ratio of gum arabic to said disperse oil phase is greater than 1:1.

23. The method of claim 22 wherein the emulsion has an oil disperse phase and wherein the weight fraction of the disperse is from 1 to 20 wt. % based on the weight of the emulsion.

24. The method of claim 22 wherein the weight fraction of the disperse oil phase in the emulsion is from 2 to 8 wt. % based on the weight of the emulsion; wherein the weight ratio of gum arabic to said disperse oil phase is greater than 3:1.

25. The method of claim 22 wherein the wherein the weight fraction of the disperse oil phase in the emulsion is from 8 to 20 wt. % based on the weight of the emulsion; wherein the weight ratio of gum arabic to said disperse oil phase is from 1:1 to 3:1.

26. The method of claim 22, wherein said cannabinoid is one or more of tetrahydrocannabinol (THC) and cannabidiol (CBD).

27. The method of claim 22, wherein the weight ratio of said cannabinoid to vegetable oil is between 1:0.1 and 1:9.
